(19) United States
(12) Patent Application Publication
(10) Pub. No.: US 2022/0168355 A1
(43) Pub. Date: Jun. 2, 2022

(54) Methods for Treating Conditions by Restoring Central Nervous System Endocrine Gland Function, and Compositions and Devices for Practicing the Same

(71) Applicant: Palo Alto Investors LP, Palo Alto, CA (US)

(72) Inventor: Anthony Joonkyoo Yun, Menlo Park, CA (US)

(21) Appl. No.: 17/672,172

(22) Filed: Feb. 15, 2022

Related U.S. Application Data
(63) Continuation of application No. 14/303,492, filed on Jun. 12, 2014.
(60) Provisional application No. 61/834,772, filed on Jun. 13, 2013.

(51) Int. Cl. A61K 35/28

(52) U.S. Cl. CPC A61K 35/28; A61N 1/3606

(57) ABSTRACT

Methods of improving a condition in a subject are provided herein. Aspects of the methods include at least partially restoring normal function of a central nervous system endocrine gland in a manner sufficient to improve the condition in the subject. In some instances, the condition is an aging associated condition. Aspects of the invention further include compositions, systems and devices for practicing the methods.

### CROSS REFERENCE TO RELATED APPLICATIONS

Pursuant to 35 U.S.C. § 119 (e), this application claims priority to the filing date of U.S. Provisional Application Ser. No. 61/834,772 filed on Jun. 13, 2013; the disclosure of which application is herein incorporated by reference.

### INTRODUCTION

There are a variety of conditions that can affect an individual's health and well-being. The treatment of various conditions that affect the health and well-being of an individual has been around for centuries. Such treatments include pharmacological, surgical, and life style (dietetic, exercise, etc.) changes. In general, the armament of treatment options available to a physician to treat such conditions has increased tremendously, especially in the last century.

However, while the number of treatment options has increased, typically such options are merely palliative, i.e., are designed for the relief of symptoms of a condition rather than actually being curative of the disorder itself. In fact, treatment protocols effectively directed at the underlying cause of a condition are quite rare.

As such, there continues to be an interest in the development of new protocol options for treating conditions. Of particular interest are protocols for treating conditions that are directed at the cause of the condition rather than the symptoms thereof.

### SUMMARY

Methods of improving a condition in a subject are provided herein. Aspects of the methods include at least partially restoring normal function of a central nervous system endocrine gland in a manner sufficient to improve the condition in the subject. In some instances, the condition is an aging associated condition. Aspects of the invention further include compositions, systems and devices for practicing the methods.

In one aspect, provided herein is a method of improving a condition in a subject, the method including the step of at least partially restoring normal function of a central nervous system endocrine gland in a manner sufficient to improve the condition in the subject. In certain embodiments, the at least partially restoring normal function step includes restoring endocrine function so that the endocrine function is closer to that of a healthy human 25 year old.

In some embodiments, the at least partially restoring endocrine function step modulates autonomic function. In certain embodiments, the autonomic function is modulated so that the sympathetic/parasympathetic bias of the subject closer to the sympathetic/parasympathetic bias of a healthy human 25 year old. In some embodiments, the at least partially restoring normal function step includes restoring pituitary function so that the pituitary function is closer to that of a healthy human 25 year old.

In some embodiments, the at least partially restoring normal function of the central nervous system endocrine gland comprises electrical stimulation. In other embodiments, the at least partially restoring normal function of the central nervous system endocrine gland includes pharmacological agent administration.

In yet other embodiments, the at least partially restoring normal function of the central nervous system endocrine gland includes a cellular therapy. In particular embodiments, the cellular therapy employs pluripotent cells. In certain embodiments, the at least partially restoring normal function of the central nervous system endocrine gland or glands includes inhibiting apoptosis of the cells of the endocrine gland or glands, the central nervous system endocrine gland or glands or in some instances non-endocrine cells, e.g., cells in some of or the entire body. In certain embodiments, the at least partially restoring normal function of the central nervous system endocrine gland or glands includes decreasing degeneration of the endocrine gland or gland cells, the central nervous system endocrine gland or glands, or non-endocrine gland cells, including cells in some of or the entire body.

In certain instances, the central nervous system endocrine gland is a hypothalamus. In other embodiments, the central nervous system endocrine gland is a pineal gland. In other embodiments, the central nervous system endocrine gland is a pituitary gland.

In some embodiments, the condition is an autonomic nervous system associated condition. In some instances, the autonomic nervous system associated condition is a sympathetic bias associated condition. In specific embodiments the sympathetic bias associated condition is an aging associated condition.

In other instances, the autonomic nervous system associated condition is a parasympathetic bias associated condition. In specific embodiments, the parasympathetic bias associated condition is a vagal bias associated condition.

In yet other embodiments, the condition being improved in the subject is an aging associated condition.

### DETAILED DESCRIPTION

Methods of improving a condition in a subject are provided herein. Aspects of the methods include at least partially restoring normal function of a central nervous system endocrine gland in a manner sufficient to improve the condition in the subject. In some instances, the condition is an aging associated condition. Aspects of the invention further include compositions, systems and devices for practicing the methods.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the invention, aspects of embodiments of methods of the invention are described first in greater detail, followed by a description of representative devices which find use in practicing various embodiments of the methods.

Methods

As summarized above, the subject methods provided herein are methods for improving a condition in a subject by at least partially restoring normal function of a central nervous system endocrine gland in a manner sufficient to improve the condition in the subject.

Any suitable condition can be treated by the subject methods. In certain embodiments, the subject methods are employed to treat a condition that can be improved by at least partially restoring normal function of a central nervous system endocrine gland. As used herein, a “central endocrine gland” is any gland located in the central nervous system (i.e., brain and spinal cord) of the subject that regulates or is involved in endocrine function including, but not limited to, the hypothalamus, the pineal gland, and the pituitary gland. “Function” and “activity” are used interchangeably herein. As used herein, “normal function” or “normal activity” with respect to a central endocrine gland refers to the function or activity of a central endocrine gland of a healthy subject of a particular age. In certain embodiments, the healthy subject is a healthy human 18 year old, 19 year old, 20 year old, 21 year old, 22 year old, 23 year old, 24 year old, 25 year old, 26 year old, 27 year old, 28 year old, 29 year old, 30 year old, 31 year old, 32 year old, 33 year old 34 year old, 35 year old, 36 year old, 37 year old, 38 year old, 39 year old, 40 year old, 41 year old, 42 year old, 43 year old, 44 year old, 45 year old, 46 year old, 47 year old, 48 year old, 49 year old or 50 year old. In some instances, the normal function with respect to a central endocrine gland is that of a healthy human 25 year old.

Restoration of Endocrine Function

In certain embodiments, the subject methods are used to improve a condition in a subject by at least partially restoring the endocrine function of one or more of the subjects' central nervous system endocrine glands. In particular instances, the method involves partially restoring the endocrine function of the subject's hypothalamus. In other instances, the method involves partially restoring the endocrine function of the subject's pituitary gland. In yet other embodiments, the method involves partially restoring the endocrine function of the subject's pineal gland.

In certain embodiments, the endocrine gland is the hypothalamus. The hypothalamus is a portion of the brain that is located below the thalamus and above the brain stem that functions to maintain homeostasis in a subject. In a subject, factors such as blood pressure, body temperature, fluid and electrolyte balance and body weight are held to a precise value called the set-point. Although this set-point can migrate over time, from day to day the hypothalamus functions to keep the set point of these factors fixed through homeostasis. To achieve homeostasis, the hypothalamus receives inputs about the state of these factors (e.g., information from vagus, spinal cord, retina, and limbic and olfactory systems) and, based on these inputs, sends neural signals to the autonomic system or endocrine signals to the pituitary. As such, the hypothalamus functions to maintain homeostasis through its control of the autonomic nervous system and endocrine system.

With respect to its endocrine function, the hypothalamus functions in controlling the pituitary gland, which in turn regulates various endocrine glands and organs. Such control of the pituitary gland occurs through two axes: 1) the hypothalamic-adenohypophyseal (anterior pituitary) axis; and 2) the hypothalamic-neurohypophyseal (posterior pituitary) axis. In some embodiments, the condition is improved by restoring hypothalamic endocrine function so that it is closer to that of a healthy human 25 year old.

In certain instances, the condition is improved by restoring hypothalamic function in the hypothalamic-adenohypophyseal (anterior pituitary) axis. In the hypothalamic-adenohypophyseal axis, hypophysiotropic hormones are released from the median eminence, itself a prolongation of the hypothalamus, into the hypophyseal portal system, which leads them to the anterior pituitary where they exert their regulatory functions on the secretion of adenohypopyseal hormones. As such, in certain instances, the subject methods are employed to treat a condition that is improved by restoring the function of the subject's hypothalamus to synthesize and/or secrete such hypophysiotropic hormones. Examples of hypophysiotropic hormones created and secreted by the hypothalamus in the hypothalamic-adenohypophyseal (anterior pituitary) axis include, but are not limited to: thyrotropin-releasing hormone (TRH, TRF, or PRH); corticotropin-releasing hormone (CRH or CRF); dopamine (DA or PIH); growth hormone-releasing hormone (GHRH); Gonadotropin-releasing hormone (GnRH or LHRH); somatostatin (SS, GHIH, or SRIF); vasopressin; oxytocin; neurotensin; or orexin.

In certain embodiments, the condition is improved by restoring hypothalamic function in the hypothalamic-neurohypophyseal (posterior pituitary) axis. In the hypothalamic-neurohypophyseal axis, neurophypophysial hormones are released from the posterior pituitary, which is a prolongation of the hypothalamus, into the circulation. In certain embodiments, the subject methods are employed to treat a condition that is improved by restoring the function of the subject's hypothalamus to synthesize and/or secrete such neurophypophysial hormones. Examples of neurophypophysial hormones created and secreted by the hypothalamus include, but are not limited to oxytocin and vasopressin.

In some instances, the subject methods are employed to improve a condition that can be improved by partially restoring the endocrine function of the subject's pituitary gland. The pituitary gland is a central nervous system endocrine gland that is regulated by the hypothalamus and is capable of secreting several different hormones that regulate homeostasis. In specific instances, the methods include partially restoring the ability of the pituitary gland to synthesize and/or secrete one or more specific hormones. The anterior pituitary synthesizes and secretes hormones under the influence of hypothalamic hormones through the hypothalamic-hypophsial portal system. Hormones synthesized and secreted by the anterior pituitary include somatotrophins (e.g., growth hormone); thyrotrophins (e.g., thyroid-stimulating hormone (TSH); corticotropins (e.g., adrenocorticotropic hormone (ACTH) and Beta-endorphin); lactotrophins (e.g., prolactic (PRL); gonadotropin (e.g, luteinizing hormone and follicle-stimulating hormone); and melanotrophin (e.g., melanocyte-stimulating hormone). Hormones stored and secreted by the posterior pituitary include oxytocin and vasopressin (antidiuretic hormone (ADH)). In some instances, the methods include partially restoring the ability of the anterior pituitary gland to synthesize and/or secrete one or more specific anterior pituitary hormones.

In other instances, the subject methods are employed to improve a condition that can be improved by partially restoring the endocrine function of the subject's pineal gland. The pineal gland produces the serotonin derivate melatonin, a hormone that affects the modulation of wake/sleep patterns and seasonal functions. In certain instances, the condition is improved by restoring the ability of the pineal gland to produce melatonin. In other instances, the condition is improved by restoring the ability of the pineal gland to regulate the hypothalamus.

Restoration of the endocrine function of a central nervous system endocrine gland so that it is at least closer to that of a healthy human (e.g., a healthy human 25 year old) can be achieved using any suitable protocol, including, but not limited to cellular therapy, electrical and/or pharmacologic protocols as described below. By “at least closer” is meant, in some instances, that the target endocrine function is restored to be 50% or more, e.g., 75% or more of the target function, such as 80% or more of the target function, including 90% or more of the target function, e.g., 95% or more of the target function, including 99% or more of the target function. Endocrine function of a restored central nervous system endocrine gland can be assayed using any suitable method including, but not limited to, immunochemiluminometric assays (see, e.g., Spencer et al. J Clin Endocrinol Metab 76 (2): 494-8 (1993) and enzyme-linked immunosorbent assays (see, e.g., Kim et al. Br J Dermatol 1555(5): 910-5 (2006).

Restoration of Autonomic Function

In other embodiments, the subject methods are employed to improve a condition that can be improved by restoring the normal function of the autonomic function of the central nervous system endocrine gland.

In some instances, the subject methods are employed to treat a condition that is caused by an abnormality in the autonomic function of the hypothalamus. In certain instances, the abnormality is a sympathetic bias mediated condition. In other instances, the abnormality is a parasympathetic bias mediated condition. Sympathetic and parasympathetic bias mediated conditions are physiological conditions having one or more undesirable symptoms, where the symptoms arise (at least in part) from sympathetic or parasympathetic bias (at least in a portion of the subject's autonomic nervous system), respectively. Sympathetic and parasympathetic bias mediated conditions include both chronic and acute conditions. In some instances, the conditions of interest are disease conditions. In some instances, the conditions of interest are conditions arising in response to one or more stimuli, e.g., ingestion of nutritional or therapeutic compositions, exposure to certain environmental conditions, infection with a pathogenic agent, induction of stress, e.g., from exercise, etc. Examples of specific conditions of interest are provided in greater detail below.

In certain embodiments of the subject method, the restoration of the central nervous system endocrine function modulates autonomic function. By “modulating” is meant altering or changing one aspect or component to provide a change, alteration or shift in another aspect or component. Modulating autonomic function is achieved by modulating at least one portion of the subject's autonomic nervous system. By “modulating at least one portion of the subject's autonomic nervous system” is meant altering or changing at least a portion of an autonomic nervous system by a means to provide a change, alteration or shift in at least one component or aspect of the autonomic nervous system.

In some instances of the subject methods, modulation of the autonomic nervous system includes modulating the parasympathetic and/or sympathetic activity in the subject. “Parasympathetic activity” refers to activity of the parasympathetic nervous system whereas “sympathetic activity” refers to activity of the sympathetic nervous system. With respect to the hypothalamus, anterior and medial hypothalamic regions exert parasympathetic effects, whereas the posterior and lateral hypothalamic regions exert sympathetic effects. Stimulation of the anterior hypothalamus (anterolateral region) excites the parasympathetic nervous system and inhibits the sympathetic nervous system. Upon such stimulation, heart and blood pressures decrease (the vagal response), the visceral vessels dilate, peristalsis and secretion of digestive juices increase, the pupils constrict and salivation increases. In contrast, stimulation of the posterior hypothalamus (posteromedial region) excites the sympathetic nervous system and inhibits the parasympathetic nervous system. Upon such stimulation, heart beat and blood pressure increases, the visceral vessels constrict, peristalsis and secretion of gastric juices decrease, pupils dilate and sweating and piloerection occur.

In some embodiments, the restoration of the central nervous system endocrine function modulates at least one of decreasing parasympathetic activity and/or increasing sympathetic activity in a subject to improve a condition caused by parasympathetic bias. In other embodiments, the restoration of the central nervous system endocrine function modulates at least one of decreasing sympathetic activity and/or increasing parasympathetic activity in a subject to improve a condition caused by sympathetic bias. Conditions that are caused by a sympathetic bias include, but are not limited to aging related diseases (e.g., cardiovascular disease, cancer, arthritis, cataracts, osteoporosis, type 2 diabetes, hypertension; shy dragers, multi-system atrophy, age related inflammation conditions and diabetes). Conditions that are caused by a parasympathetic bias include, but are not limited to an allergy, common cold eczema, asthma, anaphylaxis, attention deficit hyperactive disorder (ADHD), autism, obesity, depression, and food allergy.

Restoration of function of a central nervous system endocrine gland (e.g., hypothalamus) that also modulates the autonomic nervous system may be carried out using any suitable protocol, including, but not limited to cellular therapy, electrical and/or pharmacologic protocols as described below. The pharmacological, cellular therapeutic, and/or electrical modulation of the central nervous system endocrine gland may provide an increase in function of at least a portion of the autonomic system, e.g., increase function in at least one sympathetic or parasympathetic nerve fiber, and/or provide a decrease in function or dampening of a portion of the autonomic system, e.g., may inhibit activity in at least one sympathetic or parasympathetic nerve fiber or inhibit nerve pulse transmission.

In some instances, the modulation that is achieved in practicing methods of the invention may be quantified. One way of quantifying modulation of at least one portion of the subject's autonomic nervous system is the parasympathetic/sympathetic activity ratio. By “parasympathetic/sympathetic activity ratio” is meant the ratio of activity of the sympathetic nervous system to the activity of the parasympathetic nervous system. As such, methods according to certain embodiments include modulating a sympathetic/parasympathetic activity ratio in the subject.

In some instances, the central nervous system endocrine gland (e.g., hypothalamus) function is restored in such a manner to shift or change parasympathetic activity and/or sympathetic activity from a first state to a second state, where the second state is characterized by an increase or decrease in the sympathetic activity/parasympathetic activity ratio relative to the first state.

Accordingly, some embodiments of the subject invention include restoring the normal function of a central nervous system endocrine gland in such a manner that modulates at least a portion of a subject's autonomic nervous system to increase the sympathetic activity/parasympathetic activity ratio, i.e., to increase sympathetic activity relative to parasympathetic activity (in other words to decrease parasympathetic activity relative to sympathetic activity) so as to treat a subject for a condition that can be treated by such modulation (i.e. a condition caused by parasympathetic bias). Increasing the sympathetic activity/parasympathetic activity ratio may be achieved by stimulating the posteromedial region of the hypothalamus to increase activity in at least a portion of the sympathetic system, e.g., stimulating at least one sympathetic nerve fiber. Alternatively or in addition to stimulating at least one sympathetic nerve fiber to increase activity, increasing the sympathetic activity/parasympathetic activity ratio may be achieved by inhibiting activity in the parasympathetic system, e.g., inhibiting activity in the anterolateral hypothalamic region to achieve an increased sympathetic activity relative to parasympathetic activity. Still further, in certain embodiments the subject invention provides methods of both increasing activity in the posteromedial hypothalamic region and inhibiting activity in the anterolateral hypothalamic region to achieve the desired result.

Other embodiments of the subject invention include restoring the normal function of a central nervous system endocrine gland in such a manner that moedulates at least a portion of a subject's autonomic nervous system to decrease the sympathetic activity/parasympathetic activity ratio, i.e., to decrease sympathetic activity relative to parasympathetic activity (in other words, to increase parasympathetic activity relative to sympathetic activity) so as to treat a subject for a condition that can be treated by such modulation (i.e. a condition caused by sympathetic bias). Decreasing the sympathetic activity/parasympathetic activity ratio may be achieved by stimulating the anterolateral region of the hypothalamus to increase activity in at least a portion of the parasympathetic system, e.g., stimulating at least one parasympathetic nerve fiber. Alternatively or in addition to stimulating at least one parasympathetic nerve fiber to increase activity, decreasing the sympathetic activity/parasympathetic activity ratio may be achieved by inhibiting activity in the sympathetic system, e.g., inhibiting activity in the posteromedial hypothalamic region to achieve a decreased sympathetic activity relative to parasympathetic activity. Still further, in certain embodiments the subject invention provides methods of both increasing activity in the anterolateral hypothalamic region and inhibiting activity in the posteromedial hypothalamic region to achieve the desired result.

As will be described in greater detail below, while the ratio of sympathetic function/parasympathetic function may be modulated according to embodiments of the subject invention to treat or improve a subject for a condition (e.g., aging associated conditions) the net result may be a parasympathetic bias (i.e., a parasympathetic dominance), a sympathetic bias (i.e., sympathetic dominance), or the activities of the sympathetic system and parasympathetic system may be substantially equal (i.e., neither is dominant).

By “bias”, is meant that the particular “biased” component of the autonomic nervous system has a higher activity level than the other component. For example, a parasympathetic bias refers to a higher level of parasympathetic activity than sympathetic activity, and vice versa, where such bias may be systemic or localized. As such, by “vagal bias”, is meant that that the particular biased component of the autonomic nervous system that has a higher activity level than the other component is the vagus nerve or a portion of the autonomic nervous system associated with the vagus nerve. Vagal bias may be characterized by one or more of vagal dominance, vagal hypersensitivity and/or sympathetic insufficiency. The net result of the subject methods to treat a condition may be higher or greater sympathetic activity relative to parasympathetic activity in at least the area of the targeted autonomic system (i.e., that portion in need of modulation), or substantially equal activity levels of sympathetic activity and parasympathetic activity.

As noted above, in certain embodiments activity in at least a portion of the autonomic nervous system is increased by the restoration of a central nervous system endocrine gland function. For example, any portion of a central nervous system endocrine gland (e.g., a hypothalamus, pineal gland or pituitary gland) that is involved in the sympathetic system, e.g., one or more nerve fibers, may be stimulated to increase sympathetic activity to provide the desired ratio of parasympathetic/sympathetic activity. In other words, activity in at least a portion of a central nervous endocrine gland that is involved the sympathetic nervous system may be increased such that at least a portion of the sympathetic nervous system may be “up-regulated”. In other instances, any portion of a central nervous system endocrine gland (e.g., a hypothalamus, pineal gland or pituitary gland) that is involved in the parasympathetic system, e.g., one or more nerve fibers, may be stimulated to increase parasympathetic activity to provide the desired ratio of parasympathetic/sympathetic activity. In other words, activity in at least a portion of a central nervous endocrine gland that is involved the parasympathetic nervous system may be increased such that at least a portion of the parasympathetic nervous system may be “up-regulated”.

In certain embodiments, increasing activity in, or up-regulating, at least a part of the sympathetic system may be desired in instances where, prior to the application of autonomic nervous system-modulating agent, parasympathetic activity is higher than desired, e.g., higher than sympathetic activity (e.g., there exists a relative parasympathetic bias) and as such the subject methods may be employed to increase sympathetic activity to a level above or rather to a level that is greater than parasympathetic activity or may be employed to modulate the differential between the parasympathetic-sympathetic systems such that the result of increasing sympathetic activity may be a sympathetic bias, parasympathetic bias or may be an equalization of the two systems (i.e., the activities of the two systems are approximately equal—including equal), but the difference between the parasympathetic-sympathetic systems may be modulated, e.g., reduced or minimized or increased in certain embodiments. Accordingly, the subject methods may be employed to increase sympathetic activity above that of parasympathetic activity and/or may be employed to modulate (increase or decrease) the differential between the two systems, but in certain embodiments may be employed to decrease the parasympathetic activity/sympathetic activity ratio.

In other embodiments, increasing activity in, or up-regulating, at least a part of the parasympathetic system may be desired in instances where, prior to the application of autonomic nervous system-modulating agent, sympathetic activity is higher than desired, e.g., higher than parasympathetic activity (e.g., there exists a relative sympathetic bias) and as such the subject methods may be employed to increase parasympathetic activity to a level above or rather to a level that is greater than sympathetic activity or may be employed to modulate the differential between the parasympathetic-sympathetic systems such that the result of increasing parasympathetic activity may be a parasympathetic bias, sympathetic bias or may be an equalization of the two systems (i.e., the activities of the two systems are approximately equal—including equal), but the difference between the parasympathetic-sympathetic systems may be modulated, e.g., reduced or minimized or increased in certain embodiments. Accordingly, the subject methods may be employed to increase parasympathetic activity above that of sympathetic activity and/or may be employed to modulate (increase or decrease) the differential between the two systems, but in certain embodiments may be employed to decrease the parasympathetic activity/sympathetic activity ratio.

In certain embodiments, a parasympathetic bias may be the normal state, but the ratio of the two systems may be abnormal or otherwise contributing to a condition. Increasing sympathetic bias may also be desired in instances where, prior to the restoration of the normal function of a central nervous system endocrine gland, sympathetic activity is higher than the parasympathetic activity, but the differential between the two needs to be modulated such as increased further, e.g., the sympathetic activity is normal or above normal (i.e., abnormally high) and/or the parasympathetic activity is normal or below normal (i.e., abnormally low) or above normal (i.e., abnormally low).

For example, such instances may occur where a subject has normal or above normal sympathetic function, but also has elevated parasympathetic function. Other instances may include below normal sympathetic function, but normal or elevated parasympathetic function, etc. It may also be desirable to increase sympathetic function in instances where the respective activities of the two system are analogous or approximately equal, including equal, prior to increasing activity in the sympathetic system, but the level of one or both is abnormally high or abnormally low. The above-described examples of instances where increasing sympathetic activity may be desired is exemplary only and is in no way intended to limit the scope of the invention and other instances where increasing sympathetic activity may be desired will be apparent to those of skill in the art.

In other embodiments, a sympathetic bias may be the normal state, but the ratio of the two systems may be abnormal or otherwise contributing to a condition. Increasing parasympathetic bias may also be desired in instances where, prior to the restoration of the normal function of a central nervous system endocrine gland, parasympathetic activity is higher than the sympathetic activity, but the differential between the two needs to be modulated such as increased further, e.g., the parasympathetic activity is normal or above normal (i.e., abnormally high) and/or the sympathetic activity is normal or below normal (i.e., abnormally low) or above normal (i.e., abnormally low).

For example, such instances may occur where a subject has normal or above normal parasympathetic function, but also has elevated sympathetic function. Other instances may include below normal parasympathetic function, but normal or elevated sympathetic function, etc. It may also be desirable to increase parasympathetic function in instances where the respective activities of the two system are analogous or approximately equal, including equal, prior to increasing activity in the parasympathetic system, but the level of one or both is abnormally high or abnormally low. The above-described examples of instances where increasing parasympathetic activity may be desired is exemplary only and is in no way intended to limit the scope of the invention and other instances where increasing sympathetic activity may be desired will be apparent to those of skill in the art.

As noted above, in certain embodiments, activity in at least a portion of the central nervous system endocrine gland (e.g., the hypothalamus) that is involved in the parasympathetic or sympathetic system may be inhibited to modulate at least a portion of the autonomic nervous system. Inhibiting or “down-regulating” activity in at least a part of the autonomic nervous system, may be desired in instances where, the sympathetic or parasympathetic activity is higher than desired. For example, parasympathetic activity may be higher than the sympathetic activity (i.e., there exists a parasympathetic bias) or parasympathetic activity may be less than or approximately equal to, including equal, to sympathetic activity, and the subject methods may be employed to modulate the differential between the parasympathetic-sympathetic systems such that the net result of decreasing sympathetic activity may be a sympathetic bias, parasympathetic bias or may be an equalization of the two systems (i.e., the activities of the two systems are approximately equal—including equal), but the difference between the parasympathetic-sympathetic systems may be modulated, e.g., increased or reduced in certain embodiments. Accordingly, the subject methods may be employed to decrease parasympathetic activity below that of sympathetic activity and/or may be employed to modulate (decrease or increase) the differential between the two systems, where in certain embodiments may be employed to decrease the ratio of parasympathetic activity to sympathetic activity.

For example, decreasing activity in at least a portion of the parasympathetic system may be employed where there is a normal or an abnormally low sympathetic function and/or abnormally high parasympathetic function. Such may also be desired in instances where, prior to decreasing parasympathetic function in, e.g., at least one parasympathetic nerve fiber, sympathetic activity is higher than the parasympathetic activity, but the differential between the two needs to be increased further. For example, such instances may occur where a subject has normal or above normal (i.e., abnormally high) parasympathetic function, but also has elevated sympathetic function (i.e., abnormally high), e.g., a relative bias towards sympathetic function may be present. Other instances include normal or below normal (i.e., abnormally low) parasympathetic activity and/or normal or above normal (i.e., abnormally high) sympathetic activity. The above-described examples of instances where decreasing parasympathetic activity may be desired is exemplary only and is in no way intended to limit the scope of the invention and other instances where decreasing parasympathetic activity to provide an increase in the parasympathetic activity/sympathetic activity ratio may be desired will be apparent to those of skill in the art.

Decreasing activity in at least a portion of the sympathetic system may be employed where there is a normal or an abnormally low parasympathetic function and/or abnormally high sympathetic function. Such may also be desired in instances where, prior to decreasing sympathetic function in, e.g., at least one parasympathetic nerve fiber, parasympathetic activity is higher than the sympathetic activity, but the differential between the two needs to be increased further. For example, such instances may occur where a subject has normal or above normal (i.e., abnormally high) sympathetic function, but also has elevated parasympathetic function (i.e., abnormally high), e.g., a relative bias towards parasympathetic function may be present. Other instances include normal or below normal (i.e., abnormally low) sympathetic activity and/or normal or above normal (i.e., abnormally high) parasympathetic activity. The above-described examples of instances where decreasing sympathetic activity may be desired is exemplary only and is in no way intended to limit the scope of the invention and other instances where decreasing sympathetic activity to provide an increase in the parasympathetic activity/sympathetic activity ratio may be desired will be apparent to those of skill in the art.

One way of inhibiting activity in at least a portion of the autonomic nervous system is by the application of a nerve block. Application of a nerve block at least partially prevents nerve transmission across the location of the block. A nerve block can be administered to modulate autonomic function using all the methods and devices described herein including pharmacological and/or electrical means.

As noted above, in certain embodiments, activity in at least a portion of the autonomic nervous system may be increased and activity in at least a portion of the autonomic nervous system may be decreased. For example, in certain embodiments, activity in at least a portion of the sympathetic system may be increased and activity in at least a portion of the parasympathetic system may be inhibited, e.g., to decrease the parasympathetic activity/sympathetic activity ratio. In other embodiments, activity in at least a portion of the parasympathetic system may be increased and activity in at least a portion of the sympathetic system may be inhibited, e.g., to decrease the parasympathetic activity/sympathetic activity ratio. As described above, any portion of a central nervous system endocrine gland that is involved in the parasympathetic and/or sympathetic nervous systems may be modulated to increase activity and activity in any portion of the central nervous system endocrine gland may be inhibited to provide the desired ratio of parasympathetic activity to sympathetic activity. Such a protocol may be employed, e.g., in instances where sympathetic function is normal or abnormally low and/or parasympathetic function is normal or abnormally high, or where parasympathetic function is normal or abnormally low and/or sympathetic function is normal or abnormally high, where normal is determined by the typical or average autonomic nervous system functions for a healthy subject, e.g., a healthy human subject ranging in age from about 20 years old to about 25 years old.

Embodiments wherein activity in at least a portion of the autonomic nervous system may be increased and activity in at least a portion of the autonomic nervous system may be decreased may be employed to alter the dominance and/or may be employed to modulate the differential between the two systems. For example, prior to modulating the autonomic system according to the subject invention, the activity in the parasympathetic system may be higher than activity in the sympathetic system and the subject methods may be employed to increase the sympathetic activity to a level that is greater than the parasympathetic activity and/or may be employed to alter the differential or difference in activity levels of the two systems such as decreasing the difference in activity levels or increasing the difference in activity levels.

Increasing activity in at least a portion of the autonomic nervous system, e.g., increasing activity in at least a portion of the parasympathetic system, and decreasing activity in at least a portion of the autonomic nervous system, e.g., decreasing activity in at least a portion of the sympathetic system, may be performed simultaneously or sequentially such that at least a portion of the autonomic nervous system, e.g., at least a portion of the parasympathetic nervous system, may be pharmacologically and/or electrically modulated to increase activity therein prior or subsequent to inhibiting activity in at least a portion of the autonomic nervous system e.g., at least a portion of the sympathetic nervous system, such as by electrical and/or pharmacological means.

Regardless of whether increasing activity in at least a portion of the autonomic nervous system, e.g., in at least a portion of the parasympathetic system, and decreasing activity in at least a portion of the autonomic nervous system, e.g., in at least a portion of the sympathetic system, is performed simultaneously or sequentially, the parameters for increasing activity in at least a portion of autonomic nervous system and decreasing activity in at least a portion of the autonomic nervous system may be analogous to that described above.

Restoring function to a central nervous system endocrine gland that causes modulation of the autonomic nervous system may be accomplished using any suitable method, including employing electrical, thermal, vibrational, magnetic, acoustic, baropressure, optical, or other sources of energy to modulate autonomic balance, where in representative embodiments modulation is achieved via pharmacological modulation and/or electrical energy modulation in a manner that is effective to treat a subject for a food allergy syndrome condition. Certain embodiments include pharmacologically or electrically stimulating a central nervous system endocrine gland that causes a modulation of at least a portion of a subject's autonomic nervous system, e.g., by increasing parasympathetic activity and/or decreasing sympathetic activity or by increasing sympathetic activity and/or decreasing parasympathetic activity in at least a portion of the subject's autonomic nervous system. In certain instances, cellular therapeutic protocols can be used to modulate autonomic function. For example, parasympathetic activity can be increased by transplanting differentiated cells that are involved in parasympathetic function (e.g., neurons from the anterolateral region of the hypothalamus), whereas sympathetic activity can be increased by transplanting differentiated cells that are involved in sympathetic function (e.g., neurons from the posteromedial region of the hypothalamus). In some instances, parasympathetic activity can be increased by transplanting stem cells or progenitor cells that are capable of differentiating in vivo into cells that function in sympathetic or parasympathetic activity. In certain embodiments, modulation may include increasing the sympathetic activity/parasympathetic activity ratio in at least a portion of the subject's autonomic nervous system. In certain embodiments, a combination of electrical, pharmacological, and cellular modulation may be employed.

Cellular Modulation

In certain embodiments of the subject methods, restoring normal function of a central nervous system endocrine gland is achieved using a cellular therapy. In some instances, restoration of a function of interest (e.g., endocrine function or autonomic function) of a central nervous system endocrine gland is achieved through the transplantation of a cell to the site of the central nervous system endocrine gland, wherein the transplanted cell is subsequently capable of being incorporated into the gland and performing the function of interest.

Cells that can be transplanted include, but are not limited to: a) stem cells; b) progenitor cells; or c) stem cells or progenitor cells that have been differentiated in vitro into a particular central nervous endocrine gland cell type. In some embodiments, the transplanted cell is a pluripotent embryonic stem cell. In other embodiments, the transplanted cell is a multipotent progenitor cell. Transplanted embryonic and progenitor cells used in the subject methods are capable of proliferation and differentiation into a cell of a particular central nervous system endocrine gland. In instances where the transplanted cells are stem cells or progenitor cells, such transplanted cells are capable of proliferating and/or differentiating in vivo into cells of the endocrine gland for which normal function is to be restored. In particular embodiments, the transplanted cell is a neural stem cell or a neural progenitor cell. Stem cells and progenitor cells can be obtained from any suitable source, including, but not limited to, fetal, juvenile, or adult tissue. In particular instances, stem cells can be obtained from the cerebral cortex, cerebellum, midbrain, brainstem, spinal cord, ventricular tissues and areas of the peripheral nervous system including carotid bodies and the adrenal medulla.

In some instances, the transplanted cell is a cell of a central nervous system endocrine gland (e.g., pineal gland, hypothalamus gland, pituitary gland) that has been differentiated from a stem cell or progenitor cell in vitro in the presence of suitable biological agents, including any agents that are useful for the proliferation, differentiation or functioning of a central nervous system endocrine gland for use with the subject methods. Examples of such biological agents include, but are not limited to, growth factors (e.g., FGF-1, FGF-2, EGF, EGF-like ligands, TGF, IGF-1, NGF, PDGF, and TGFβs); trophic factors (e.g., BDNF, CNTF, and glial-derived neutrotrophic factor (GFNF)); regulators of intracellular pathways associated with growth factor activity, interleukins, Bcl-2 gene product, bone morphogenic protein (BMP-2), macrophage inflammatory proteins (MIP-1α, MIP-1β and MIP-2); antisense nucleotides against transcripts for EGF receptors, FGF receptors, and the like; heparin-like molecules, amphiregulin, retinoic acid, and TNFα.

In certain embodiments of the subject methods wherein restoration of endocrine function is desired, the transplanted cells are cells capable of secreting a particular hormone. In instances wherein hypothalamic endocrine function is restored, transplanted cells include, but are not limited to: parvocellular neurosecretory cells of the paraventricular nucleus (thyrotropin-releasing hormone and corticotropin-releasing hormone producing cells); dopamine neurons of the arcuate nucleus (dopamine producing cells); neuroendocrine neurons of the arcuate nucleus (growth hormone-releasing hormone producing cells); neuroendocrine cells of the preoptic area (gonadotropin-releasing hormone producing cells); neuroendocrine cells of the periventricular nucleus (somatostatin producing cells); and magnocellular neurosecretory cells of the paraventricular nucleus and supraoptic nucleus (oxytocin and vasopressing producing cells) or stem cells or progenitor cells that are capable of differentiating into such cell types. In instances wherein hypothalamic autonomic function is restored, the transplanted cells include neurons from the anterolateral region of the hypothalamus that function in parasympathetic activity or neurons from the posteromedial region of the hypothalamus that function in sympathetic activity or stem cells or progenitor cells capable of differentiating into such cells. In other instances, the transplanted cell is a cell of a pineal gland capable of producing melatonin or a stem cell or progenitor cell capable of differentiating into such a cell. In other embodiments, the transplanted cell is an anterior pituitary cell capable of synthesizing and/or secreting a pituitary hormone or a stem cell or progenitor cell capable of differentiating into such a cell.

The transplanted cell can further be genetically modified in any manner that is useful for restoring the normal function of the central nervous endocrine gland. For example, wherein the transplanted cell is a differentiated hypothalamic cell or a stem cell or progenitor cell that can differentiate into a hypothalamic cell, such transplanted cell may be genetically modified to include a nucleic acid encoding a hypothalamic hormone, including any of the hypothalamic hormones described above. Wherein the transplanted cell is a differentiated pituitary cell or a stem cell or progenitor cell that can differentiate into a pituitary cell, such transplanted cell may be genetically modified to include a nucleic acid encoding a pituitary hormone, including any of the pituitary hormones described above.

In certain embodiments, the transplanted cells are capable of differentiating and proliferating faster than the rate of apoptosis of the cells of or degeneration of the subject's central endocrine nervous system gland. In some instances, the particular therapeutic approach may result in at least a reduction of apoptosis and/or degeneration of cells in the body apart from the central endocrine nervous system, including cells throughout the body.

Protocols for differentiation, proliferation and transplantation of neural cells are further described in U.S. Pat. Nos. 6,040,180; 6,497,872; 8,318,704; International Publication No. WO 2012/016409; Wataya et al. PNAS 105(33): 11796-11801 (2008), the disclosures of which are herein incorporated by reference.

Pharmacologic Modulation

As noted above, in certain embodiments of the subject methods, restoration of a central nervous system endocrine gland is achieved by pharmacologic modulation of the central nervous system endocrine gland. By “pharmacologically modulating a central nervous system endocrine gland” is meant altering or changing a central nervous system endocrine gland by pharmacological means to provide a change, alteration or shift in at least one or more of its functions (e.g., endocrine function or autonomic function). In embodiments in which pharmacological agent is administered, any suitable protocol may be used, where certain protocols include using an pharmacological agent administering device to deliver a suitable amount of pharmacological agent to a subject. Methods and corresponding devices and systems for applying at least one pharmacological agent to a subject and which may be adapted for use in the subject invention are described, e.g., in U.S. Pat. Nos. 7,363,076; 7,149,574, U.S. patent application Ser. Nos. 10/661,368; 10/748,976; 10/871,366; 10/846,486; 10/917,270; 10/962,190; 11/060,643 11/251,629; 11/238,108; 11/592,027; 60/654,139; and 60/702,776; the disclosures of which are herein incorporated by reference.

Any convenient pharmacological agent may be employed. In certain embodiments, the pharmacological agent is a hypothalamic hormone or analogue thereof. Hypothalamic hormones include, but are not limited to: GnRH agonists (e.g., Buserelin (suprefact), gonadorelin (Factrel), Goserelin (Zoladex), Histrelin (Supprelin), Leuprolide (Lurpon, Lurpon Depot, Viadur), Nafarelin (synarel) and Triptorelin (Trelstar Depot, Trelstar LA); GNRH antagonoists (e.g., Abarelix (Plenaxis), Cetrorelix (Cetrotide), and Ganirelix (Antagon); and dopamine agonists (Bromocriptine (Parlodel), Cabergoline (Dostinex). In other instances, the

Pro-sympathetic agents of interest include, but are not limited to: beta agonists, e.g., dobutamine, metaproterenol, terbutaline, ritodrine, albuterol; alpha agonists, e.g., selective alpha 1-adrenergic blocking agents such as phenylephrine, metaraminol, methoxamine; prednisone and steroids, (e.g., available under the brand names CORATN, DELTASONE, LIQUID PRED, MEDICORTEN, ORASONE, PANASOL-S, PREDNICEN-M, PREDNISONE INTENSOL); indirect agents that include norepinephrine, e.g., ephedrine, ampthetamines, phenylpropanolamines, cyclopentamines, tuaminoheptanes, naphazolines, tetrahydrozolines; epinephrine; norepinephrine; acetylcholine; sodium; calcium; angiotensin I; angiotensin II; angiotensin converting enzyme I (“ACE I”); angiotensin converting enzyme II (“ACE II”); aldosterone; potassium channel blockers and magnesium channel blockers, e.g., valproate (sodium valproate, valproic acid), lithium; cocaine; amphetamines; terbutaline; dopamine; doputamine; antidiuretic hormone (“ADH”) (also known as vasopressin); oxytocin (including PITOCINE); THC cannabinoids; and combinations thereof.

Pro-parasympathetic agents of interest include, but are not limited to: Beta Blockers, Aldosterone Antagonists; Angiotensin II Receptor Blockers; Angiotensin Converting Enzyme Inhibitors; Statins; Triglyceride Lowering Agents; Insulin Sensitizers; Insulin Secretagogues; Insulin Analogs; Alpha-glucosidase Inhibitors; SGLT2 Inhibitors; Immunomodulators, including agents that bind/react to CD4, gp39, B7, CD19, CD20, CD22, CD401, CD40, CD40L and CD23 antigens; Sympathomimetics; Cholinergics; Calcium Channel Blockers; Sodium Channel Blockers; Glucocorticoid Receptor Blockers; Peripheral Adrenergic Inhibitors; Blood Vessel Dilators; Central Adrenergic Agonists; Alpha-adrenergic Blockers; Combination Diuretics; Potassium-sparing Diuretics; Nitrate Pathway Modulators; Cyclic Nucleotide Monophosphodiesterase (PDE) Inhibitors; Vasopressin Inhibitors; Renin Inhibitors; Estrogen and Estrogen Analogues and Metabolites; Vesicular Monoamine Transport (VMAT) Inhibitors; Progesterone Inhibitors; Testosterone Inhibitors; Gonadotropin-releasing Hormone Inhibitors; Dipeptidyl Peptidase IV inhibitors; Anticoagulants; Thrombolytics.

Agents of interest further include anti-apoptotic agents. Such agents may be any agents which at least slow, if not completely stop, apoptosis in a cell. Apoptotic agents of interest may be general apoptotic agents which act on cells of the entire body, or specific or a certain portion of the body, e.g., one or more central nervous system endocrine claims. Examples of apoptotic agents include, but are not limited to those compositions described in U.S. Pat. Nos. 8,143,300 and 6,596,693; the disclosures of which are herein incorporated by reference.

Electrical Modulation

In certain embodiments, to accomplish the modulation of at least a portion of a subject's central nervous endocrine gland function (e.g., autonomic function or endocrine function), electrical energy (electrical modulation) may be applied to at least a portion of a subject's central nervous system endocrine gland, where such electrical energy may be excitatory or inhibitory and in certain embodiments may include both excitatory and inhibitory stimulation. By “electrically modulating at least a portion of a subject's autonomic nervous system” is meant altering or changing at least a portion of an autonomic nervous system by electrical means to provide a change, alteration or shift in at least one component or aspect of the function of the central nervous system endocrine gland (e.g., autonomic function or endocrine function). Embodiments of the subject methods may also, in addition to electrical energy, include administering at least one pharmacological agent (pharmacological modulation) to said subject to modulate at least a portion of a subject's central nervous system endocrine gland.

Any suitable area may be targeted for electrical modulation. Areas that may be targeted include, but are not limited to, pre- and post-ganglionic nerve fibers, as well as ganglionic structures, efferent and afferent nerve fibers, synapses, etc., and combinations thereof in certain embodiments. In certain embodiments, activity in a given nerve fiber may be electrically modulated in more than one area of the nerve fiber. In certain embodiments, electrical energy is applied to modulate synaptic efficiency. In certain embodiments, electrical energy is applied using any of the devices described below.

A number of different methods and corresponding devices and systems for applying electrical energy to a subject and which may be adapted for use in the subject invention are described, e.g., in U.S. Pat. Nos. 7,149,574; 7,711,430; and 7,363,076; as well as U.S. patent application Ser. No. 11/592,027; the disclosures of which are herein incorporated by reference.

Activation of the electrical energy supplying device directly applies the electrical output of the device, i.e., electrical energy, to the targeted area. For example, electrodes may be positioned to direct electrical impulses to specific nerve fibers, etc. The exact parameters of the protocol may vary depending on the particular subject, condition being treated, etc. An electronic current wave may be provided when the electrical energy is applied. In certain embodiments, the current wave includes current waves of high frequency, e.g., high frequency pulses, where the current wave may also include low frequency amplitude modulation. In certain embodiments, a plurality of high frequency bursts of current pulses may be applied in addition to the application of underlying low frequency continuous stimulus. Monopolar or multipolar technologies may be employed.

For example, to increase activity in a portion of the autonomic nervous system, voltage or intensity may range from about 1 millivolt to about 1 volt or more, e.g., 0.1 volt to about 50 volts, e.g., from about 0.2 volt to about 20 volts and the frequency may range from about 1 Hz to about 2500 Hz, e.g., about 1 Hz to about 1000 Hz, e.g., from about 2 Hz to about 100 Hz in certain embodiments. In certain embodiments pure d-c voltages may be employed. The pulse width may range from about 1 microsecond to about 2000 microseconds or more, e.g., from about 10 microseconds to about 2000 microseconds, e.g., from about 15 microseconds to about 1000 microseconds, e.g., from about 25 microseconds to about 1000 microseconds. The electrical output may be applied for at least about 1 millisecond or more, e.g., about 1 second, e.g., about several seconds, where in certain embodiments the stimulation may be applied for as long as about 1 minute or more, e.g., about several minutes or more, e.g., about 30 minutes or more may be used in certain embodiments.

In some instances where an electrical protocol is employed, the target condition is not a bronchoconstriction condition, such as asthma, e.g., as described in United States Patent Application 20120004701.

Paradoxical Modulation

In some instances, the methods include employing a paradoxical protocol in order to obtain a desired increase or decrease in sympathetic/parasympathetic activity ratio. In some of these embodiments, the central nervous system endocrine is restored in a manner such that the sympathetic/parasympathetic activity ratio is increased initially in a manner effective to cause the subject to mount a compensatory response effective to ultimately decrease the sympathetic/parasympathetic activity ratio. In other embodiments, the central nervous system endocrine is restored in a manner such that the sympathetic/parasympathetic activity ratio is decreased initially in a manner effective to cause the subject to mount a compensatory response effective to ultimately increase the sympathetic/parasympathetic activity ratio. In certain embodiments, the magnitude of increase or decrease in the sympathetic/parasympathetic activity ratio is two-fold or greater, e.g., 5-fold or greater.

In practicing the subject methods, the sympathetic/parasympathetic activity ratio is decreased by applying an appropriate stimulus to the subject, where the stimulus is of a nature and magnitude sufficient to achieve the desired enhancement. In certain embodiments, the applied stimulus is one of short duration, where by short duration is meant that the applied stimulus lasts for less than about 1 week, e.g., less than about 3 days, e.g., less than about 1 day, e.g., less than about 12 hours, where the duration of the applied stimulus may be even shorter. Where the stimulus is a pharmacological stimulus, the duration refers to the period in which the pharmacological agent from an administered dosage is active. Where the stimulus is an electrical stimulus, the duration refers to the total of electrical applications received by a subject over a given period, analogous to a dose of a pharmacological agent.

Following decrease of the sympathetic/parasympathetic activity ratio via an applied stimulus, as described above, the stimulus is removed, e.g., by metabolization of the pharmacological agent or cessation of application of electrical energy, and the subject is permitted to mount a compensatory response. In this following period, no additional stimulus is administered to the subject. The duration of this period between stimulus application, which may be referred to as a “holiday” period, may vary, but in representative embodiments is 1 day or longer, such as 2 days or longer, including 5 days or longer, 10 days or longer, e.g., 15 days or longer. As such, embodiments of the methods include non-chronic (i.e., non-continuous) application of the stimulus, e.g., non-chronic administration of a pharmacologic agent.

In certain embodiments, the methods include close monitoring or supervision of the subject during and/or after application of the stimulus. This monitoring may be completely automated, or at least in part performed manually, e.g., by a health care professional. For example, a health care professional can closely watch the subject following application of the stimulus as well as during the holiday period following stimulus application, and based on this monitoring determine when a next stimulus should be applied. Monitoring also assures that the symptom enhancement is not so severe as to be ultimately damaging to the subject at an unacceptable level. Certain aspects of the monitoring may be automated. For example, following administration, the subject may enter one or more physiological parameters into an automated system, which uses the input parameters to automatically determine whether the subject is staying within a predetermined set of physiological parameters, or whether intervention is necessary. In certain embodiments, the automated monitoring system may also be integrated with a stimulus application device, such that the system, based on monitored parameters, determines when next to administer a stimulus, the duration of the next stimulus, etc. As such, the method may be characterized as applying a first stimulus to the subject and monitoring the subject for a response thereto. Following this first step, the method further includes applying at least a second stimulus to the subject, wherein the second stimulus is determined based on the monitored response to the first stimulus.

In certain embodiments, wherein a decrease in the sympathetic/parasympathetic activity ratio is ultimately desired, the sympathetic/parasympathetic activity ratio is initially increased by applying a stimulus, followed by removal of the stimulus to allow for a compensatory decrease in sympathetic/parasympathetic activity ratio.

In certain embodiments, stimulus to the subject is done in an “irregularly irregular” manner. As such, duration of the stimulus application events, as well as duration of holiday periods between such events, varies randomly over the entire course of a treatment, or at least a portion thereof. In addition, the variation does not follow any pattern, but instead is random.

In practicing the subject methods, the applied stimulus may vary, where in certain embodiments the stimulus may be a pharmacological stimulus and/or an electrical stimulus. As such, in certain embodiments, the stimulus is a pharmacological stimulus. In other embodiments, the stimulus is an electrical stimulus. In yet other embodiments, the stimulus is a combination of pharmacological and electrical stimuli. Accordingly, in certain embodiments, the enhancing is by administering a pharmacological agent to the subject. In yet other embodiments, the enhancing is by electrical stimulation, e.g., by employing an implanted electrical energy application device.

Representative pharmacological agents that may find use in certain embodiments of the subject invention include both pro-parasympathetic and sympathetic agents. Pro parasympathetic agents of interest include, but are not limited to: Beta Blockers, Aldosterone Antagonists; Angiotensin II Receptor Blockers; Angiotensin Converting Enzyme Inhibitors; Statins; Triglyceride Lowering Agents; Insulin Sensitizers; Insulin Secretagogues; Insulin Analogs; Alpha-glucosidase Inhibitors; SGLT2 Inhibitors; Immunomodulators, including agents that bind/react to CD4, gp39, B7, CD19, CD20, CD22, CD401, CD40, CD40L and CD23 antigens; Sympathomimetics; Cholinergics; Calcium Channel Blockers; Sodium Channel Blockers; Glucocorticoid Receptor Blockers; Peripheral Adrenergic Inhibitors; Blood Vessel Dilators; Central Adrenergic Agonists; Alpha-adrenergic Blockers; Combination Diuretics; Potassium-sparing Diuretics; Nitrate Pathway Modulators; Cyclic Nucleotide Monophosphodiesterase (PDE) Inhibitors; Vasopressin Inhibitors; Renin Inhibitors; Estrogen and Estrogen Analogues and Metabolites; Vesicular Monoamine Transport (VMAT) Inhibitors; Progesterone Inhibitors; Testosterone Inhibitors; Gonadotropin-releasing Hormone Inhibitors; Dipeptidyl Peptidase IV inhibitors; Anticoagulants; Thrombolytics. Pro-sympathetic agents of interest include, but are not limited to: beta agonists, e.g., dobutamine, metaproterenol, terbutaline, ritodrine, albuterol; alpha agonists, e.g., selective alpha 1-adrenergic blocking agents such as phenylephrine, metaraminol, methoxamine; prednisone and steroids, (e.g., available under the brand names CORATN, DELTASONE, LIQUID PRED, MEDICORTEN, ORASONE, PANASOL-S, PREDNICEN-M, PREDNISONE INTENSOL); indirect agents that include norepinephrine, e.g., ephedrine, ampthetamines, phenylpropanolamines, cyclopentamines, tuaminoheptanes, naphazolines, tetrahydrozolines; epinephrine; norepinephrine; acetylcholine; sodium; calcium; angiotensin I; angiotensin II; angiotensin converting enzyme I (“ACE I”); angiotensin converting enzyme II (“ACE II”); aldosterone; potassium channel blockers and magnesium channel blockers, e.g., valproate (sodium valproate, valproic acid), lithium; cocaine; amphetamines; terbutaline; dopamine; doputamine; antidiuretic hormone (“ADH”) (also known as vasopressin); oxytocin (including PITOCINE); THC cannabinoids; and combinations thereof.

Instead of, or in addition to, pharmacological protocols, electrical protocols may be employed in these paradoxical approaches. In such instances, an electrical protocol is employed to obtain the desired paradoxical decrease or increase in sympathetic/parasympathetic activity ratio. As reviewed above, a number of different methods and corresponding devices and systems for applying electrical energy to a subject and which may be adapted for use in the subject invention are described, e.g., in U.S. Pat. Nos. 7,149,574; 7,711,430; and 7,363,076; as well as U.S. patent application Ser. No. 11/592,027; the disclosures of which are herein incorporated by reference.

Subjects

The methods described herein may be employed with a variety of different types of subjects, i.e., animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

In some embodiments, the subject in which central nervous system endocrine gland function has been restored has been diagnosed as having a sympathetic or parasympathetic bias mediated condition. In some instances, the methods may include diagnosing the subject as having a sympathetic or parasympathetic bias mediated condition. Diagnoses of such conditions may be made using any convenient protocol. In some instances, the subject is also one that has been determined to have an autonomic dysfunction. As used herein, the term “autonomic dysfunction” describes any disease or malfunction of the autonomic nervous system. Specific types of autonomic dysfunction of interest include sympathetic and parasympathetic bias. A specific type of parasympathetic bias of interest is vagal bias.

In certain embodiments modulation of at least a portion of a subject's autonomic nervous system is not performed unless one or more aspects of the autonomic nervous system are detected and indicate such modulation is necessary. One aspect that may indicate modulation is necessary is the existence of an autonomic dysfunction, such as parasympathetic bias, e.g. vagal bias.

In certain embodiments the subject has an autonomic dysfunction before diagnosis of an autonomic dysfunction (e.g., sympathetic or parasympathetic bias) occurs. Any suitable physical and/or chemical aspect or indicator of the autonomic nervous system may be employed by one or more of a doctor, nurse, medical professional or individual with appropriate expertise to diagnose an autonomic dysfunction.

An autonomic dysfunction in a subject may be tested for by detecting one or more aspects of the autonomic nervous system, e.g., amounts of T helper cells (Th1 and/or Th2), conduction, catecholamine levels, heart rate variability (“HRV”), action potentials, QT interval, particular hormone levels, as well as chronotropic, inotropic, and vasodilator responses. For example, in certain embodiments HRV measures such as low frequency peak (“LF”), high frequency peak (“HF”), and the LF/HF ratio may be used as indicators of different aspects of the autonomic nervous system. In certain embodiments, detection may include detecting the activity or function of a particular organ or system under the control of the autonomic nervous system. Any suitable detection means may be employed to detect relevant information about the autonomic nervous system.

These and other methods and devices for detecting one or more aspects of the autonomic nervous system potentially indicating an autonomic dysfunction that may be employed by embodiments of the subject methods include those described in U.S. Pat. Nos. 7,899,527 and 6,490,480 and U.S. patent Ser. Nos. 10/861,566 and 12/727,560.

Utility

The subject methods find use in a variety of applications in which it is desired to treat a subject for a condition, e.g., a condition that can be improved by at least partially restoring normal function of a central nervous system endocrine gland. In such methods, at least a portion of a subject's central nervous system endocrine gland (e.g., hypothalamus, pineal gland, or pituitary gland) is restored to normal function in a manner sufficient to improve the condition in the subject. As discussed herein, in some instances the function of the central nervous system endocrine is restored in a manner such that endocrine function of the central nervous system endocrine gland. In other instances, the function of the central nervous system endocrine is restored in such a manner that causes the modulation of autonomic function in the subject, e.g., in a manner to increase the parasympathetic activity/sympathetic activity ratio or decrease the parasympathetic activity/sympathetic activity ratio.

The subject methods find use in the treatment of a variety of different conditions. By treatment is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the condition, or at least symptoms associated therewith, are completely inhibited, e.g. prevented from happening, or stopped, e.g. terminated, such that the subject no longer suffers from the condition, or at least the symptoms that characterize the condition. In certain embodiments, the condition being treated is a disease condition.

In some instances, the method is for the treatment of an aging associated condition. In some instances, aging associated conditions are due in part to sympathetic bias. As such, methods provided herein wherein the restoration of a central nervous system endocrine gland modulates sympathetic/parasympathetic bias in the subject is believed to improve such age associated conditions. Aging-associated conditions include, but are not limited to, cardiovascular disease, cancer, arthritis, cataracts, osteoporosis, type 2 diabetes, hypertension; shy dragers, multi-system atrophy, age related inflammation conditions and diabetes.

In some instances, the condition is a cardiovascular condition. Cardiovascular conditions include, but are not limited to, cardiovascular disease, e.g., atherosclerosis, coronary artery disease, hypertension, hyperlipidemia, eclampsia, pre-eclampsia, cardiomyopathy, volume retention, congestive heart failure, QT interval prolongation, aortic dissection, aortic aneurysm, arterial aneurysm, arterial vasospasm, myocardial infarction, reperfusion syndrome, ischemia, sudden adult death syndrome, arrhythmia, fatal arrythmias, coronary syndromes, coronary vasospasm, sick sinus syndrome, bradycardia, tachycardia, thromboembolic disease, deep vein thrombosis, coagulopathy, disseminated intravascular coagulation (“DIC”), mesenteric ischemia, syncope, venous thrombosis, arterial thrombosis, malignant hypertension, secondary hypertension, primary pulmonary hypertension, secondary pulmonary hypertension, raynaud's, paroxysmal supraventricular tachycardia, and the like.

In certain embodiments, the subject method is for the treatment of a cancer. Cancers include, but are not limited to, bladder cancer, breast cancer, colon cancer, rectal cancer, endometrial cancer, kidney cancer, leukemia, lung cancer, melanoma, non-Hodgkin lymphoma, pancreatic cancer, prostate cancer and thyroid cancer, and the like.

In some instances, the subject method is for the treatment of arthritis. Arthritic diseases include, but are not limited to, osteoarthritis, rheumatoid arthritis, gout, pseudo-gout, septic arthritis, ankylosing spondylitis, juvenile idiopathic arthritis and Still's disease.

In some instances, the method is for the treatment of an endocrine condition. Endocrine conditions including endocrine diseases, e.g., hypothyroidism, hyperglycemia, diabetes, obesity, syndrome X, insulin resistance, polycystic ovarian syndrome (“PCOS”), and the like.

In other instances, the subject method is for the treatment of a neurodegenerative condition. Neurodegenerative conditions include neurodegenerative diseases, e.g., Alzheimer's Disease, Pick's Disease, Parkinson's Disease, dementia, delirium, amyotrophic lateral sclerosis, and the like.

In yet other instances, the method is for the treatment of a neuroninflammatory condition. Neuroinflammatory conditions include, but are not limited to neuroinflammatory diseases, e.g., viral meningitis, viral encephalitis, fungal meningitis, fungal encephalitis, multiple sclerosis, charcot joint, schizophrenia, myasthenia gravis, and the like.

In some instances, the method is for the treatment of an orthopedic inflammatory condition. Orthopedic inflammatory conditions include orthopedic inflammatory diseases, e.g., osteoarthritis, inflammatory arthritis, regional idiopathic osteoporosis, reflex sympathetic dystrophy, Paget's disease, osteoporosis, antigen-induced arthritis, juvenile chronic arthritis, and the like.

In other instances, the method is for the treatment of a lymphoproliferative condition. Lymphoproliferative conditions include lymphoproliferative diseases, e.g., lymphoma, lymphoproliferative disease, Hodgkin's disease, inflammatory pseudomotor of the liver, and the like.

In yet other instances, the method is for the treatment of an autoimmune condition. Autoimmune conditions include automimmune diseases, e.g., Graves disease, raynaud's, hashimoto's, takayasu's disease, kawasaki's diseases, arteritis, scleroderma, CREST syndrome, allergies, dermatitis, Henoch-schlonlein purpura, goodpasture syndrome, autoimmune thyroiditis, myasthenia gravis, Reiter's disease, lupus, and the like.

In some embodiments, the method is for the treatment of an inflammatory condition. Inflammatory conditions include, but are not limited to acute respiratory distress syndrome (“ARDS”), multiple sclerosis, rheumatoid arthritis, juvenile rheumatoid arthritis, juvenile chronic arthritis, migraines, chronic headaches, and the like.

In other embodiments, the method is for the treatment of an infection disease. Infectious diseases included, but are not limited to sepsis, viral and fungal infections, diseases of wound healing, wound healing, tuberculosis, infection, AIDS, human immunodeficiency virus, and the like.

In yet other embodiments, the method is for the treatment of a pulmonary condition. Pulmonary conditions include, but are not limited to pulmonary diseases, e.g., tachypnea, fibrotic lung diseases such as cystic fibrosis and the like, interstitial lung disease, desquamative interstitial pneumonitis, non-specific interstitial pneumonitis, intrapulmonary shunts; lymphocytic interstitial pneumonitis, usual interstitial pneumonitis, idiopathic pulmonary fibrosis, pulmonary edema, aspiration, asphyxiation, pneumothorax, right-to-left shunts, left-to-right shunts, respiratory failure, and the like.

In some instances, the method is for the treatment of a transplant-related condition. Transplant-related conditions include transplant related side effects such as transplant rejection, transplant-related tachycardia, transplant related renal failure, transplant related bowel dysmotility, transplant-related hyperreninemia, and the like.

In other instances, the method is for the treatment of a gastrointestinal condition. Gastrointestinal conditions include, but are not limited to, gastrointestinal diseases, e.g., hepatitis, xerostomia, bowel mobility, peptic ulcer disease, constipation, ileus, irritable bowel syndrome, post-operative bowel dysmotility, inflammatory bowel disease, typhilitis, cholelethiasis, cholestasis, fecal incontinence, cyclic vomiting syndrome, and the like.

In yet other instances, the method is for the treatment of a genitourinary condition. Genitourinary conditions including genitourinary diseases, e.g., bladder dysfunction, renal failure, erectile dysfunction, hyperreninemia, hepatorenal syndrome, pulmonary renal syndrome, incontinence, arousal disorder, menopausal mood disorder, premenstrual mood disorder, renal tubular acidosis, pulmonary renal syndrome, and the like.

In other instances, the method is for the treatment of a skin condition, including a skin disease such as wrinkles, cutaneous vasculitis, psoriasis, rash; and the like.

In other embodiments, the method is for the treatment of a neurologic condition, including, but not limited to, a neurologic disease such as epilepsy, depression, schizophrenia, seizures, stroke, insomnia, cerebral vascular accident, transient ischemic attacks, stress, bipolar disorder, concussions, post-concussive syndrome, cerebral vascular vasospasm, central sleep apnea, obstructive sleep apnea, sleep disorders, headaches including chronic headaches, migraines, acute disseminated encephalomyelitis (“ADEM”), and the like.

In yet other embodiments, the method is for the treatment of a pediatric condition, including a pediatric diseases such as a respiratory distress syndrome, sudden infant death syndrome, hirschsprung disease, bronchopulmonary dysplasia, congenital megacolon, ananglionosis, juvenile rheumatoid arthritis, juvenile chronic arthritis, and the like.

In some embodiments, the method is for the treatment of a Th-2 dominant condition such as typhilitis, osteoporosis, lymphoma, myasthenia gravis, lupus, and the like.

In some embodiments, the condition is a disease that causes hypoxia, hypercarbia, hypercapnia, acidosis, acidemia, Chronic Obstructive Pulmonary Disease (“COPD”), emphysema, any chronic lung disease that causes acidosis, acute pulmonary embolism, sudden adult death syndrome (“SADS”), chronic pulmonary embolism, pleural effusion, cardiogenic pulmonary edema, non-cardiogenic pulmonary edema, acute respiratory distress syndrome (ARDS), neurogenic edema, hypercapnia, acidemia, asthma, renal tubular, asthma, acidosis, chronic lung diseases that cause hypoxia, hypercarbia or hypercapnia, and the like.

In other embodimetns, the condition is an OB-GYN conditions including, but not limited to, an OB-GYN diseases such as amniotic fluid embolism, menopausal mood disorders, premenstrual mood disorders, pregnancy-related arrhythmias, fetal stress syndrome, fetal hypoxia, amniotic fluid embolism, gestational diabetes, pre-term labor, cervical incompetence, fetal distress, peri-partum maternal mortality, peripartum cardiomyopathy, labor complications, premenstrual syndrome, dysmenorrheal, endometriosis, and the like.

In yet other embodiments, the subject method is for the treatment of a sudden death syndrome (e.g., sudden adult death syndrome, sudden infant death syndrome, and the like); a menstrual related disorders (e.g., pelvic pain, dysmenorrheal, gastrointestinal disease, nausea, and the like); a peripartum or pregnancy related condition (e.g., peripartum cardiomyopathy, and the like); a fibrosis; a post-operative recovery conditions (e.g., post-operative pain, post operative ileus, post-operative fever, post-operative nausea, and the like); a post-procedural recovery condition (e.g., post-procedural pain, post procedural ileus, post-procedural fever, post-procedural nausea, and the like); a chronic pain; a trauma; hospitalization; glaucoma; male infertility; a disorder of thermoregulation; respiratory sinus arrhythmia; VQ mismatch; or a fibromyalgia; and the like.

Other conditions may also be treated in accordance with the subject invention. Embodiments of the subject invention include treating one or more conditions, sequentially or at the same time, in accordance with the subject invention.

In some embodiments, the restoration of the central nervous system endocrine function modulates at least one of decreasing parasympathetic activity and/or increasing sympathetic activity in a subject to improve a condition caused by parasympathetic bias. In other embodiments, the restoration of the central nervous system endocrine function modulates at least one of decreasing sympathetic activity and/or increasing parasympathetic activity in a subject to improve a condition caused by sympathetic bias. Conditions that are caused by a sympathetic bias and may be treated by methods such as described herein include, but are not limited to aging related diseases (e.g., cardiovascular disease, cancer, arthritis, cataracts, osteoporosis, type 2 diabetes, hypertension; shy dragers, multi-system atrophy, age related inflammation conditions and diabetes). Conditions that are caused by a parasympathetic bias and may be treated by methods such as described herein include, but are not limited to an allergy, common cold eczema, asthma, anaphylaxis, attention deficit hyperactive disorder (ADHD), autism, obesity, depression, and food allergy. It is noted that parasympathetic or vagal bias mediated conditions, e.g., autism, may also be treated via methods such as described in U.S. patent application Ser. No. 14/012,771, the disclosure of which is herein incorporated by reference. An embodiment of the invention includes treating autism with methods such as described in U.S. patent application Ser. No. 14/012,771 (the disclosure of which methods is incorporated herein by reference), which may or may not be used in conjunction with restoration of central nervous system endocrine gland function, e.g., as describe elsewhere herein.

Devices

A number of different devices and systems may be employed in accordance with the subject invention. Devices and systems which may be adapted for use in the subject invention include devices and systems for applying at least one pharmacological agent to a subject and devices and systems for applying electrical energy to a subject.

Devices and Systems for Applying Pharmacological Agent(s)

Different devices and systems for applying one or more pharmacological agents to a subject which may be adapted for use in the subject invention include embodiments configured to deliver pharmacological agent(s) using any of the methods described above. A device for applying one or more pharmacological agents to modulate autonomic function is a “pharmacological modulator”.

Embodiments may include an implantable or external pharmacological delivery device such as, but not limited to, pumps, epidural injectors, syringes or other injection apparatus, catheter and/or reservoir operatively associated with a catheter, etc. For example, in certain embodiments a delivery device employed to deliver at least one pharmacological agent to a subject may be a pump, syringe, catheter or reservoir operably associated with a connecting device such as a catheter, tubing, or the like. Containers suitable for delivery of at least one pharmacological agent to a pharmacological agent administration device include instruments of containment that may be used to deliver, place, attach, and/or insert the at least one pharmacological agent into the delivery device for administration of the pharmacological agent to a subject and include, but are not limited to, vials, ampules, tubes, capsules, bottles, syringes and bags.

In some embodiments, the device for applying one or more pharmacological agents includes a sensor for detecting a food allergy syndrome, condition, symptom and/or instigator. As used herein, an “instigator” is an aspect that causes or aggravates a food allergy syndrome condition and/or symptom. A sensor may take the form of an electrode or the like and may be configured specifically to detect one or more symptoms of a food allergy condition. Signals received by such a sensor may be amplified before further processing. A sensor may also take the form of a device capable of detecting nerve compound action potentials or may take the form of a transducer that includes an electrode with an ion selective coating applied which is capable of directly transducing the amount of a particular transmitter substance or its breakdown by-products. More detailed descriptions of sensors that may be employed in the practice of the subject invention, and other examples of sensors that may be employed are disclosed in U.S. Pat. No. 5,716,377, which is incorporated herein by reference. Systems for applying at least one pharmacological agent according to the methods described above are made up of one or more of the devices or components listed or incorporated by reference herein.

Devices and systems for applying at least one pharmacological agent to a subject and which may be adapted for use in the subject invention are described, e.g., in U.S. Pat. Nos. 7,363,076; 6,503,532; 5,302,395; 5,262,165; 5,248,501; 5,232,702; 5,230,896; 5,227,169; 5,212,199; 5,202,125; 5,173,302; 5,154,922; 5,139,786; 5,122,383; 5,023,252; 4,978,532; 5,324,521; 5,306,503; 5,302,395; 5,296,230; 5,286,491; 5,252,334; 5,248,501; 5,230,896; 5,227,169; 5,212,199; 5,202,125; 5,173,302; 5,171,576; 5,139,786; 5,133,972; 5,122,383; 5,120,546; 5,118,509; 5,077,054; 5,066,494; 5,049,387; 5,028,435; 5,023,252; 5,000,956; 4,911,916; 4,898,734; 4,883,669; 4,882,377; 4,840,796; 4,818,540; 4,814,173; 4,806,341; 4,789,547; 4,786,277; 4,702,732; 4,690,683; 4,627,429; 4,585,452; U.S. patent application Ser. Nos. 10/748,897; 10/748,976; 10/871,366; 10/846,486 10/917,270; 10/962,190; 11/060,643 11/251,629; 11/238,108; 11/592,027; 60/654,139; 60/702,776; and elsewhere, the disclosures of which are herein incorporated by reference.

Devices and Systems for Applying Electrical Energy

Devices and systems for applying electrical energy to a subject which may be adapted for use in the subject invention include embodiments configured to deliver electrical energy using any of the methods described above. In accordance with the subject methods to apply electrical energy to a subject, once operatively positioned, the electric energy applying device is activated to provide an electrical signal to the targeted area in a manner effective to practice the subject methods.

A device for applying electrical energy to modulate autonomic function is an “electrical modulator”. Electrical modulators may be positioned directly on a targeted area and may be implantable within the body of the subject or be wholly or partially external to the subject's body. An electrical energy applying device or system typically includes a stimulator such as one or more electrodes, a controller or programmer and one or more connectors for connecting the stimulating device to the controller.

The one or more electrodes employed in the subject invention are controllable to provide output signals that may be varied in voltage, frequency, pulse width, current and intensity. The energy source for the electrical output may be provided by a battery or generator that is operatively connected to the electrode(s). The energy source may be positioned in any suitable location such as adjacent to the electrode(s), or a remote site in or on the subject's body or away from the subject's body in a remote location and the electrode may then be connected to the remotely positioned energy source using wires. A controller or programmer may also be coupled with an electric energy applying device. The programmer is typically one or more microprocessors under the control of a suitable software program.

In some embodiments, the device for applying electrical energy includes a sensor for detecting a food allergy syndrome condition symptom and/or instigator. As used herein, an “instigator” is an aspect that causes or aggravates a food allergy syndrome condition and/or symptom. A sensor may take the form of an electrode or the like and may be configured specifically to detect one or more symptoms of a food allergy condition. Signals received by such a sensor may be amplified before further processing. A sensor may also take the form of a device capable of detecting nerve compound action potentials or may take the form of a transducer that includes an electrode with an ion selective coating applied which is capable of directly transducing the amount of a particular transmitter substance or its breakdown by-products. More detailed descriptions of sensors that may be employed in the practice of the subject invention, and other examples of sensors that may be employed are disclosed in U.S. Pat. No. 5,716,377, which is incorporated herein by reference. Systems for applying electrical energy according to the methods described above are made up of one or more of the devices or components listed or incorporated by reference herein.

In embodiments in which electrical energy is used, any suitable protocol may be used, where certain protocols include using an electric energy applying device to deliver a suitable amount of electrical energy to a subject. Once an electric energy applying device is positioned in a suitable position on or about one or more targeted areas electrical energy is applied thereto for a period of time sufficient to provide the desired effect.

A number of different devices and systems for applying electrical energy to a subject and which may be adapted for use in the subject invention are described, e.g., in U.S. Pat. Nos. 7,149,574; 7,711,430; 7,363,076; U.S. patent application Ser. Nos. 10/661,368; 10/748,976; 10/871,366; 10/846,486 10/917,270; 10/962,190; 11/060,643 11/251,629; 11/238,108; 11/592,027;

60/654,139; and 60/702,776; and elsewhere, the disclosures of which are herein incorporated by reference.

All publications and patent applications mentioned in this specification are herein incorporated by reference to the same extent as if each individual publication or patent application was specifically and individually indicated to be incorporated by reference.

The invention now being fully described, it will be apparent to one of skill in the art that many changes and modifications can be made thereto without departing from the spirit and scope of the appended claims.

1. A method of improving a condition in a subject, the method comprising:
at least partially restoring normal function of a central nervous system endocrine gland in a manner sufficient to improve the condition in the subject.

2. The method according to claim 1, wherein the at least partially restoring normal function comprises restoring endocrine function so that the endocrine function is closer to that of a healthy human 25 year old.

3. The method according to claim 1, wherein the at least partially restoring endocrine function modulates autonomic function.

4. The method according to claim 3, wherein the autonomic function is modulated so that the sympathetic/parasympathetic bias of the subject is closer to the sympathetic/parasympathetic bias of a healthy human 25 year old.

5. The method according to claim 1, wherein the at least partially restoring normal function comprises restoring pituitary function so that the pituitary function is closer to that of a healthy human 25 year old.

6. (canceled)

7. The method according to claim 1, wherein the at least partially restoring normal function of the central nervous system endocrine gland comprises pharmacological agent administration.

8. The method according to claim 1, wherein the at least partially restoring normal function of the central nervous system endocrine gland comprises a cellular therapy.

9. The method according to claim 8, wherein the cellular therapy employs pluripotent cells.

10. The method according to claim 1, wherein the at least partially restoring normal function of the central nervous system endocrine gland comprises inhibiting apoptosis of cells of the endocrine gland.

11. The method according to claim 10, wherein the method further comprises inhibiting apoptosis of non-endocrine gland cells.

12. The method according to claim 1, wherein the at least partially restoring normal function of the central nervous system endocrine gland comprises decreasing degeneration of the endocrine gland.

13. The method according to claim 12, wherein the method further comprises decreasing degeneration of non-endocrine gland cells.

14. The method according to claim 1, wherein the central nervous system endocrine gland is a hypothalamus.

15. The method according to claim 1, wherein the central nervous system endocrine gland is a pineal gland.

16. The method according to claim 1, wherein the condition is an autonomic nervous system associated condition.

17. The method according to claim 16, wherein the autonomic nervous system associated condition is a sympathetic bias associated condition.

18. The method according to claim 17, wherein the sympathetic bias associated condition is an aging associated condition.

19. The method according to claim 16, wherein the autonomic nervous system associated condition is a parasympathetic bias associated condition.

20. The method according to claim 19, wherein the parasympathetic bias associated condition is a vagal bias associated condition.

21. The method according to claim 1, wherein the condition is an aging associated condition.

22. (canceled)
